SEQUENCE LISTING (2 sequences)

SEQ ID NO: 1 - DNA, 1595 bp, Homo sapiens
  CDS (14)..(1588)
ccccaccgccgccatggccaccttgctccgcagcaagctgtccaacgtggccacgtccgtgtccaacaagtcccaggccaagatgagcggcatgttcgccaggatgggttttcaggcggccacggatgaggaggcggtgggcttcgcgcattgcgacgacctcgactttgagcaccgccagggcctgcagatggacatcctgaaagccgagggagagccctgcggggacgagggcgctgaagcgcccgtcgagggagacatccattatcagcgaggcagcggagctcctctgccgccctccggctccaaggaccaggtgggaggtggtggcgaattcgggggccacgacaagcccaaaatcacggcgtgggaggcaggctggaacgtgaccaacgccatccagggcatgttcgtgctgggcctaccctacgccatcctgcacggcggctacctggggttgtttctcatcatcttcgccgccgttgtgtgctgctacaccggcaagatcctcatcgcgtgcctgtacgaggagaatgaagacggcgaggtggtgcgcgtgcgggactcgtacgtggccatagccaacgcctgctgcgccccgcgcttcccaacgctgggcggccgagtggtgaacgtagcgcagatcatcgagctggtgatgacgtgcatcctgtacgtggtggtgagtggcaacctcatgtacaacagcttcccggggctgcccgtgtcgcagaagtcctggtccattatcgccacggccgtgctgctgccttgcgccttccttaagaacctcaaggccgtgtccaagttcagtctgctgtgcactctggcccacttcgtcatcaatatcctggtcatagcctactgtctatcgcgggcgcgcgactgggcctgggagaaggtcaagttctacatcgacgtcaagaagttccccatctccattggcatcatcgtgttcagctacacgtctcagatcttcctgccttcgctggagggcaatatgcagcagcccagcgagttccactgcatgatgaactggacgcacatcgcagcctgcgtgctcaagggcctcttcgcgctcgtcgcctacctcacctgggccgacgagaccaaggaggtcatcacggataacctgcccggctccatccgcgccgtggtcaacatctttctggtggccaaggcgctgttgtcctatcctctgccattctttgccgctgtcgaggtgctggagaagtcgctcttccaggaaggcagccgcgcctttttcccggcctgctacagcggcgacgggcgcctgaagtcctgggggctgacgctgcgctgcgcgctcgtcgtcttcacgctgctcatggccatttatgtgccgcacttcgcgctgctcatgggcctcaccggcagcctcacgggcgccggcctctgtttcttgctgcccagcctctttcacctgcgcctgctctggcgcaagctgctgtggcaccaagtcttcttcgacgtcgccatcttcgtcatcggcggcatctgcagcgtgtccggcttcgtgcactccctcgagggcctcatcgaagcctaccgaaccaacgcggaggactagggcg

SEQ ID NO: 2 - protein, 525 aa, Homo sapiens
MATLLRSKLSNVATSVSNKSQAKMSGMFARMGFQAATDEEAVGFAHCDDLDFEHRQGLQMDILKAEGEPCGDEGAEAPVEGDIHYQRGSGAPLPPSGSKDQVGGGGEFGGHDKPKITAWEAGWNVTNAIQGMFVLGLPYAILHGGYLGLFLIIFAAVVCCYTGKILIACLYEENEDGEVVRVRDSYVAIANACCAPRFPTLGGRVVNVAQIIELVMTCILYVVVSGNLMYNSFPGLPVSQKSWSIIATAVLLPCAFLKNLKAVSKFSLLCTLAHFVINILVIAYCLSRARDWAWEKVKFYIDVKKFPISIGIIVFSYTSQIFLPSLEGNMQQPSEFHCMMNWTHIAACVLKGLFALVAYLTWADETKEVITDNLPGSIRAVVNIFLVAKALLSYPLPFFAAVEVLEKSLFQEGSRAFFPACYSGDGRLKSWGLTLRCALVVFTLLMAIYVPHFALLMGLTGSLTGAGLCFLLPSLFHLRLLWRKLLWHQVFFDVAIFVIGGICSVSGFVHSLEGLIEAYRTNAED

(19) United States
(12) Patent Application Publication
(10) Pub. No.: US 2002/0076758 A1
(43) Pub. Date: Jun. 20, 2002

(54) Polypeptide

(76) Inventors: Georg Christian Terstappen, Verona (IT); Cinzia Felicita Sala, Verona (IT)

Correspondence Address: DAVID J LEVY, CORPORATE INTELLECTUAL PROPERTY, GLAXOSMITHKLINE, FIVE MOORE DR., PO BOX 13398, DURHAM, NC, 27709-3398, US

(21) Appl. No.: 09/871,503

(22) Filed: May 31, 2001

(30) Foreign Application Priority Data
May 31, 2000 (GB) 0013239.9

(51) Int. Cl. G01N 33/53; C07K 17/00; A61K 38/00; C07H 21/04; C07K 14/00; C12N 15/74; A61K 39/44; C12N 15/09; C12P 21/06; C12N 15/63; A61K 39/395; C12N 15/00; A61K 39/40; A61K 39/42; C12N 5/02; C12N 15/70; C12N 5/00; C07K 1/00

(52) U.S. Cl. 435/69.1; 530/350; 536/23.5; 424/180.1; 514/12; 435/7.1; 435/320.1

(57) ABSTRACT

The present invention provides an isolated neurotransmitter transporter polypeptide. Also provided are a polynucleotide encoding a neurotransmitter transporter polypeptide and methods for identifying modulators of a neurotransmitter transporter polypeptide. Such modulators can be useful in the treatment of psychiatric and neurological/neurodegenerative disorders.

### CROSS-REFERENCE TO RELATED APPLICATION

The present invention claims priority from application number GB0013239.9 filed Jun. 1, 2000.

### FIELD OF THE INVENTION

The present invention relates to neurotransmitter transporter polypeptides.

### BACKGROUND OF THE INVENTION

In most systems termination of chemical neurotransmission is achieved by rapid uptake of the released neurotransmitter by specific neurotransmitter transporters into the synaptic terminal or the surrounding glial cells. At least two distinct superfamilies of transporters can be distinguished: the plasma membrane transporters that operate on the plasma membrane of neuronal and glial cells, and the vesicular membrane transporters that function in the uptake of neurotransmitters into synaptic vesicles. The superfamily of plasma membrane transporters can be further divided into two families depending on their ionic dependence: Na⁺/Cl⁻-dependent transporters and Na⁺/K⁺-dependent transporters.

The gene products of the Na⁺/Cl⁻ transporter family are highly conserved. In mammals they can be grouped into subfamilies: monoamine transporters, amino acid transporters and “orphan transporters”. The subfamilies with known substrates are distinguished not only by their sequence homology, but also by their substrate specificity and pharmacology, whereas for “orphan transporters” the transported substrates to date are unknown. The Na⁺/Cl⁻ transporters show a common structure of 12 transmembrane domains (12TM) with a single large loop in the external face of the membrane with potential glycosylation sites. The orphan transporter structure deviates from the norm by having two potential glycosylated loops outside the membrane.

The Na⁺/K⁺-dependent transporters function on the plasma membranes especially in excitatory amino acid (eg. glutamate) transport. The family members display 6 to 10 hydrophobic transmembrane domains and share no sequence homology with the Na⁺/Cl⁻-dependent carrier family. Hydropathic analysis of the primary sequences of vesicular monoamine and acetylcholine transporters predict 12 putative transmembrane segments, while vesicular inhibitory amino acid transporters predict 10 transmembrane domains.

Rat VGAT is homologue to the C. elegans gene unc-47. The protein has 10 transmembrane domains and colocalizes with synaptic vesicles. The gene is specifically expressed by GABA neurons. It is expressed at particularly high levels within the neocortex, hippocampus, cerebellum, striatum and thalamus.

### SUMMARY OF THE INVENTION

A novel neurotransmitter transporter polypeptide, referred to herein as HIPHUM 0000057, is now provided. This novel neurotransmitter transporter polypeptide is a screening target for the identification and development of novel pharmaceutical agents, including modulators of neurotransmitter transporter polypeptide activity. These agents may be used in the treatment and/or prophylaxis of disorders such as psychiatric disorders, bipolar disorders, unipolar depression, anxiety, schizophrenia, psychotic disorders and neurological/neurodegenerative disorders and drug dependence.

Accordingly, the present invention provides an isolated neurotransmitter transporter polypeptide comprising

(i) the amino acid sequence of SEQ ID NO: 2;

(ii) a variant thereof which has vesicular inhibitory amino acid transporter activity; or

(iii) a fragment of (i) or (ii) which has vesicular inhibitory amino acid transporter activity.

According to another aspect of the invention there is provided a polynucleotide encoding a polypeptide of the invention which polynucleotide includes a sequence comprising:

(a) the nucleic acid sequence of SEQ ID NO: 1 and/or a sequence complementary thereto;

(b) a sequence which hybridises under stringent conditions to a sequence as defined in (a);

(c) a sequence that is degenerate as a result of the genetic code to a sequence as defined in (a) or (b); or

(d) a sequence having at least 60% identity to a sequence as defined in (a), (b) or (c).

The invention also provides:

an expression vector which comprises a polynucleotide of the invention and which is capable of expressing a polypeptide of the invention;

a host cell comprising an expression vector of the invention;

a method of producing a polypeptide of the invention which method comprises maintaining a host cell of the invention under conditions suitable for obtaining expression of the polypeptide and isolating the said polypeptide;

an antibody specific for a polypeptide of the invention;

a method for identification of a substance that modulates neurotransmitter transporter activity and/or expression, which method comprises contacting a polypeptide, polynucleotide, expression vector or host cell of the invention with a test substance and determining the effect of the test substance on the activity and/or expression of the said polypeptide or the polypeptide encoded by the said polynucleotide, thereby to determine whether the test substance modulates neurotransmitter transporter activity and/or expression;

a compound which modulates neurotransmitter transporter activity and which is identifiable by the method referred to above;

a method of treating a subject having a disorder that is responsive to neurotransmitter transporter stimulation or modulation, which method comprises administering to said subject an effective amount of substance of the invention; and

use of a substance that stimulates or modulates neurotransmitter transporter activity in the manufacture of a medicament for the treatment or prophylaxis of a disorder that is responsive to stimulation or modulation of neurotransmitter transporter activity.

Preferably the disorder is selected from psychiatric disorders and neurological/neurodegenerative disorders.

### BRIEF DESCRIPTION OF THE DRAWINGS

FIG. 1 shows expression of HIPHUM 0000057 in normal tissues, as indicated in the graph.

FIG. 2 shows expression of HIPHUM 0000057 in normal and disease tissues, as labeled. Normal tissues are labeled “Normal” and disease tissues are labeled with the particular disease, in addition to the tissue type.

FIG. 3 shows expression of HIPHUM 0000057 in different areas of the brain, as labeled.

### DETAILED DESCRIPTION OF THE INVENTION

Throughout the present specification and the accompanying claims the words “comprise” and “include” and variations such as “comprises”, “comprising”, “includes” and “including” are to be interpreted inclusively. That is, these words are intended to convey the possible inclusion of other elements or integers not specifically recited, where the context allows.

The present invention relates to a human neurotransmitter transporter polypeptide, referred to herein as HIPHUM 0000057, and variants thereof. Sequence information for HIPHUM 0000057 is provided in SEQ ID NO: 1 (nucleotide) and in SEQ ID NO: 2 (amino acid). A polypeptide of the invention thus consists essentially of the amino acid sequence of SEQ ID NO: 2 or of a variant of that sequence, or of a fragment of either thereof.

Polypeptides of the invention may be in a substantially isolated form. It will be understood that the polypeptide may be mixed with carriers or diluents which will not interfere with the intended purpose of the polypeptide and still be regarded as substantially isolated. A polypeptide of the invention may also be in a substantially purified form, in which case it will generally comprise the polypeptide in a preparation in which more than 50%, e.g. more than 80%, 90%, 95% or 99%, by weight of the polypeptide in the preparation is a polypeptide of the invention. Routine methods, can be employed to purify and/or synthesise the proteins according to the invention. Such methods are well understood by persons skilled in the art, and include techniques such as those disclosed in Sambrook et al, Molecular Cloning: a Laboratory Manual, 2^{nd} Edition, CSH Laboratory Press, 1989, the disclosure of which is included herein in its entirety by way of reference.

The term “variant” refers to a polypeptide which has a same essential character or basic biological functionality as HIPHUM 0000057. The essential character of HIPHUM 0000057 can be defined as follows: HIPHUM 0000057 is a neurotransmitter transporter polypeptide. Preferably a variant polypeptide is one which binds to the same ligand or substrate as HIPHUM 0000057. Preferably the polypeptide has vesicular inhibitory amino acid transporter activity, i.e. preferably the polypeptide is capable of transporting amino acids which act as inhibitory neurotransmitters. A polypeptide having a same essential character as HIPHUM 0000057 may be identified by monitoring for a function of the neurotransmitter transporter such as vesicular inhibitory amino acid transporter activity. A full length variant is preferably one which includes ten transmembrane domains. A variant receptor may be identified by looking for substrate binding or transport. Possible neurotransmitter ligands include GABA, nipecoid acid and N-butyric acid. Preferably the ligand is GABA.

Typically, polypeptides with more than about 65% identity preferably at least 80%, at least 90%, at least 95% or at least 97% and particularly preferably at least 98% or at least 99% identity, with the amino acid sequence of SEQ ID NO: 2, are considered as variants of the proteins. Such variants may include allelic variants and the deletion, modification or addition of single amino acids or groups of amino acids within the protein sequence, as long as the peptide maintains a basic biological functionality of the HIPHUM 0000057 polypeptide.

Amino acid substitutions may be made, for example from 1, 2 or 3 to 10, 20 or 30 substitutions. The modified polypeptide generally retains activity as a neurotransmitter transporter. Conservative substitutions may be made, for example according to the following Table. Amino acids in the same block in the second column and preferably in the same line in the third column may be substituted for each other.

| | | |
|---|---|---|
| ALIPHATIC | Non-polar | G A P |
| | | I L V |
| | Polar-uncharged | C S T M |
| | | N Q |
| | Polar-charged | D B |
| | | K R |
| AROMATIC | | H F W Y |

Shorter polypeptide sequences are within the scope of the invention. For example, a peptide of at least 20 amino acids or up to 50, 60, 70, 80, 100, 150 or 200 amino acids in length is considered to fall within the scope of the invention as long as it demonstrates a basic biological functionality of HIPHUM 0000057. In particular, but not exclusively, this aspect of the invention encompasses the situation when the protein is a fragment of the complete protein sequence and may represent a ligand-binding region (N-terminal extracellular domain) or an effector binding region (C-terminal intracellular domain). Such fragments can be used to construct chimeric polypeptides preferably with another ten transmembrane polypeptide, more preferably with another member of the family of neurotransmitter transporter polypeptides. Such fragments of HIPHUM 0000057 or a variant thereof can also be used to raise anti-HIPHUM 0000057 antibodies. In this embodiment the fragment may comprise an epitope of the HIPHUM 0000057 polypeptide and may otherwise not demonstrate the substrate or ligand binding or other properties of HIPHUM 0000057.

Polypeptides of the invention may be chemically modified, e.g. post-translationally modified. For example, they may be glycosylated or comprise modified amino acid residues. They may also be modified by the addition of histidine residues to assist their purification or by the addition of a signal sequence to promote insertion into the cell membrane. Such modified polypeptides fall within the scope of the term “polypeptide” of the invention.

The invention also includes nucleotide sequences that encode for HIPHUM 0000057 or variant thereof as well as nucleotide sequences which are complementary thereto. The nucleotide sequence may be RNA or DNA including genomic DNA, synthetic DNA or cDNA. Preferably the nucleotide sequence is a DNA sequence and most preferably, a cDNA sequence. Nucleotide sequence information is provided in SEQ ID NO: 1. Such nucleotides can be isolated from human cells or synthesised according to methods well known in the art, as described by way of example in Sambrook et al, 1989.

Typically a polynucleotide of the invention comprises a contiguous sequence of nucleotides which is capable of hybridizing under selective conditions to the coding sequence or the complement of the coding sequence of SEQ ID NO: 1.

A polynucleotide of the invention can hydridize to the coding sequence or the complement of the coding sequence of SEQ ID NO: 1 at a level significantly above background. Background hybridization may occur, for example, because of other cDNAs present in a cDNA library. The signal level generated by the interaction between a polynucleotide of the invention and the coding sequence or complement of the coding sequence of SEQ ID NO: 1 is typically at least 10 fold, preferably at least 100 fold, as intense as interactions between other polynucleotides and the coding sequence of SEQ ID NO: 1. The intensity of interaction may be measured, for example, by radiolabelling the probe, e.g. with ³²P. Selective hybridisation may typically be achieved using conditions of medium to high stringency. However, such hybridisation may be carried out under any suitable conditions known in the art (see Sambrook et al, 1989. For example, if high stringency is required suitable conditions include from 0.1 to 0.2× SSC at 60° C. up to 65° C. If lower stringency is required suitable conditions include 2× SSC at 60° C.

The coding sequence of SEQ ID NO: 1 may be modified by nucleotide substitutions, for example from 1, 2 or 3 to 10, 25, 50 or 100 substitutions. The polynucleotide of SEQ ID NO: 1 may alternatively or additionally be modified by one or more insertions and/or deletions and/or by an extension at either or both ends. A polynucleotide may include one or more introns, for example may comprise genomic DNA. Additional sequences such as signal sequences which may assist in insertion of the polypeptide in a cell membrane may also be included. The modified polynucleotide generally encodes a polypeptide which has a HIPHUM 0000057 polypeptide activity. Alternatively, a polynucleotide encodes a ligand-binding portion of a polypeptide or a polypeptide which inhibits HIPHUM 0000057 activity. Degenerate substitutions may be made and/or substitutions may be made which would result in a conservative amino acid substitution when the modified sequence is translated, for example as shown in the Table above.

A nucleotide sequence which is capable of selectively hybridizing to the complement of the DNA coding sequence of SEQ ID NO: 1 will generally have at least 60%, at least 70%, at least 80%, at least 90%, at least 95%, at least 98% or at least 99% sequence identity to the coding sequence of SEQ ID NO: 1 over a region of at least 20, preferably at least 30, for instance at least 40, at least 60, more preferably at least 100 contiguous nucleotides or most preferably over the full length of SEQ ID NO: 1.

For example the UWGCG Package provides the BESTFIT program which can be used to calculate homology (for example used on its default settings) (Devereux et al (1984) Nucleic Acids Research 12, p387-395). The PILEUP and BLAST algorithms can be used to calculate homology or line up sequences (typically on their default settings), for example as described in Altschul (1993) J. Mol. Evol. 36:290-300; Altschul et al (1990) J. Mol. Biol. 215:403-10.

Software for performing BLAST analyses is publicly available through the National Centre for Biotechnology Information (http://www.ncbi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighbourhood word score threshold (Altschul et al, 1990). These initial neighbourhood word hits act as seeds for initiating searches to find HSPs containing them. The word hits are extended in both directions along each sequence for as far as the cumulative alignment score can be increased. Extensions for the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a word length (W) of 11, the BLOSUM62 scoring matrix (see Henikoff and Henikoff (1992) Proc. Natl. Acad. Sci. USA 89: 10915-10919) alignments (B) of 50, expectation (E) of 10, M=5, N=4, and a comparison of both strands.

The BLAST algorithm performs a statistical analysis of the similarity between two sequences; see e.g., Karlin and Altschul (1993) Proc. Natl. Acad. Sci. USA 90: 5873-5787. One measure of similarity provided by the BLAST algorithm is the smallest sum probability (P(N)), which provides an indication of the probability by which a match between two nucleotide or amino acid sequences would occur by chance. For example, a sequence is considered similar to another sequence if the smallest sum probability in comparison of the first sequence to the second sequence is less than about 1, preferably less than about 0.1, more preferably less than about 0.01, and most preferably less than about 0.001.

Any combination of the above mentioned degrees of sequence identity and minimum sizes may be used to define polynucleotides of the invention, with the more stringent combinations (i.e. higher sequence identity over longer lengths) being preferred. Thus, for example a polynucleotide which has at least 90% sequence identity over 25, preferably over 30 nucleotides forms one aspect of the invention, as does a polynucleotide which has at least 95% sequence identity over 40 nucleotides.

The nucleotides according to the invention have utility in production of the proteins according to the invention, which may take place in vitro, in vivo or ex vivo. The nucleotides may be involved in recombinant protein synthesis or indeed as therapeutic agents in their own right, utilised in gene therapy techniques. Nucleotides complementary to those encoding HIPHUM 0000057, or antisense sequences, may also be used in gene therapy.

Polynucleotides of the invention may be used as a primer, e.g. a PCR primer, a primer for an alternative amplification reaction, a probe e.g. labelled with a revealing label by conventional means using radioactive or non-radioactive labels, or the polynucleotides may be cloned into vectors.

Such primers, probes and other fragments will preferably be at least 10, preferably at least 15 or at least 20, for example at least 25, at least 30 or at least 40 nucleotides in length. They will typically be up to 40, 50, 60, 70, 100 or 150 nucleotides in length. Probes and fragments can be longer than 150 nucleotides in length, for example up to 200, 300, 400, 500, 600, 700 nucleotides in length, or even up to a few nucleotides, such as five or ten nucleotides, short of the coding sequence of SEQ ID NO: 1.

The present invention also includes expression vectors that comprise nucleotide sequences encoding the proteins or variants thereof of the invention. Such expression vectors are routinely constructed in the art of molecular biology and may for example involve the use of plasmid DNA and appropriate initiators, promoters, enhancers and other elements, such as for example polyadenylation signals which may be necessary, and which are positioned in the correct orientation, in order to allow for protein expression. Other suitable vectors would be apparent to persons skilled in the art. By way of further example in this regard we refer to Sambrook et al. 1989.

Polynucleotides according to the invention may also be inserted into the vectors described above in an antisense orientation in order to provide for the production of antisense RNA. Antisense RNA or other antisense polynucleotides may also be produced by synthetic means. Such antisense polynucleotides may be used as test compounds in the assays of the invention or may be useful in a method of treatment of the human or animal body by therapy.

Preferably, a polynucleotide of the invention or for use in the invention in a vector is operably linked to a control sequence which is capable of providing for the expression of the coding sequence by the host cell, i.e. the vector is an expression vector. The term “operably linked” refers to a juxtaposition wherein the components described are in a relationship permitting them to function in their intended manner. A regulatory sequence, such as a promoter, “operably linked” to a coding sequence is positioned in such a way that expression of the coding sequence is achieved under conditions compatible with the regulatory sequence.

The vectors may be for example, plasmid, virus or phage vectors provided with a origin of replication, optionally a promoter for the expression of the said polynucleotide and optionally a regulator of the promoter. The vectors may contain one or more selectable marker genes, for example an ampicillin resistance gene in the case of a bacterial plasmid or a resistance gene for a fingal vector. Vectors may be used in vitro, for example for the production of DNA or RNA or used to transfect or transform a host cell, for example, a mammalian host cell. The vectors may also be adapted to be used in vivo, for example in a method of gene therapy.

Promoters and other expression regulation signals may be selected to be compatible with the host cell for which expression is designed. For example, yeast promoters include S. cerevisiae GAL4 and ADH promoters, S. pombe nmt1 and adh promoter. Mammalian promoters include the metallothionein promoter which can be induced in response to heavy metals such as cadmium. Viral promoters such as the SV40 large T antigen promoter or adenovirus promoters may also be used. All these promoters are readily available in the art.

Mammalian promoters, such as P-actin promoters, may be used. Tissue-specific promoters are especially preferred. Viral promoters may also be used, for example the Moloney murine leukaemia virus long terminal repeat (MMLV LTR), the rous sarcoma virus (RSV) LTR promoter, the SV40 promoter, the human cytomegalovirus (CMV) IE promoter, adenovirus, HSV promoters (such as the HSV IE promoters), or HPV promoters, particularly the HPV upstream regulatory region (URR). Viral promoters are readily available in the art.

The vector may further include sequences flanking the polynucleotide giving rise to polynucleotides which comprise sequences homologous to eukaryotic genomic sequences, preferably mammalian genomic sequences, or viral genomic sequences. This will allow the introduction of the polynucleotides of the invention into the genome of eukaryotic cells or viruses by homologous recombination. In particular, a plasmid vector comprising the expression cassette flanked by viral sequences can be used to prepare a viral vector suitable for delivering the polynucleotides of the invention to a mammalian cell. Other examples of suitable viral vectors include herpes simplex viral vectors and retroviruses, including lentiviruses, adenoviruses, adeno-associated viruses and HPV viruses. Gene transfer techniques using these viruses are known to those skilled in the art. Retrovirus vectors for example may be used to stably integrate the polynucleotide giving rise to the polynucleotide into the host genome. Replication-defective adenovirus vectors by contrast remain episomal and therefore allow transient expression.

The invention also includes cells that have been modified to express the HIPHUM 0000057 polypeptide or a variant thereof. Such cells include transient, or preferably stable higher eukaryotic cell lines, such as mammalian cells or insect cells, using for example a baculovirus expression system, lower eukaryotic cells, such as yeast or prokaryotic cells such as bacterial cells. Particular examples of cells which may be modified by insertion of vectors encoding for a polypeptide according to the invention include mammalian HEK293T, CHO, HeLa and COS cells. Preferably the cell line selected will be one which is not only stable, but also allows for mature glycosylation and cell surface expression of a polypeptide. Expression may be achieved in transformed oocytes. A polypeptide of the invention may be expressed in cells of a transgenic non-human animal, preferably a mouse. A transgenic non-human animal expressing a polypeptide of the invention is included within the scope of the invention. A polypeptide of the invention may also be expressed in Xenopus laevis oocytes, in particular for use in an assay of the invention.

According to another aspect, the present invention also relates to antibodies, specific for a polypeptide of the invention. Such antibodies are for example useful in purification, isolation or screening methods involving immunoprecipitation techniques or, indeed, as therapeutic agents in their own right.

Antibodies may be raised against specific epitopes of the polypeptides according to the invention. Such antibodies may be used to block ligand binding to the polypeptide. An antibody, or other compound, “specifically binds” to a protein when it binds with preferential or high affinity to the protein for which it is specific but does substantially bind not bind or binds with only low affinity to other proteins. A variety of protocols for competitive binding or immunoradiometric assays to determine the specific binding capability of an antibody are well known in the art (see for example Maddox et al, J. Exp. Med. 158, 1211-1226, 1993). Such immunoassays typically involve the formation of complexes between the specific protein and its antibody and the measurement of complex formation.

Antibodies of the invention may be antibodies to human polypeptides or fragments thereof. For the purposes of this invention, the term “antibody”, unless specified to the contrary, includes fragments which bind a polypeptide of the invention. Such fragments include Fv, F(ab′) and F(ab′)₂ fragments, as well as single chain antibodies. Furthermore, the antibodies and fragment thereof may be chimeric antibodies, CDR-grafted antibodies or humanised antibodies.

Antibodies may be used in a method for detecting polypeptides of the invention in a biological sample, which method comprises:

I providing an antibody of the invention;

II incubating a biological sample with said antibody under conditions which allow for the formation of an antibody-antigen complex; and

III determining whether antibody-antigen complex comprising said antibody is formed.

A sample may be for example a tissue extract, blood, serum and saliva. Antibodies of the invention may be bound to a solid support and/or packaged into kits in a suitable container along with suitable reagents, controls, instructions, etc. Antibodies may be linked to a revealing label and thus may be suitable for use in methods of in vivo HIPHUM 0000057 imaging.

Antibodies of the invention can be produced by any suitable method. Means for preparing and characterising antibodies are well known in the art, see for example Harlow and Lane (1988) “Antibodies: A Laboratory Manual”, Cold Spring Harbor Laboratory Press, Cold Spring Harbor, N.Y. For example, an antibody may be produced by raising antibody in a host animal against the whole polypeptide or a fragment thereof, for example an antigenic epitope thereof, herein after the “immunogen”.

A method for producing a polyclonal antibody comprises immunising a suitable host animal, for example an experimental animal, with the immunogen and isolating immunoglobulins from the animal's serum. The animal may therefore be inoculated with the immunogen, blood subsequently removed from the animal and the IgG fraction purified.

A method for producing a monoclonal antibody comprises immortalising cells which produce the desired antibody. Hybridoma cells may be produced by fusing spleen cells from an inoculated experimental animal with tumour cells (Kohler and Milstein (1975) Nature 256, 495-497).

An immortalized cell producing the desired antibody may be selected by a conventional procedure. The hybridomas may be grown in culture or injected intraperitoneally for formation of ascites fluid or into the blood stream of an allogenic host or immunocompromised host. Human antibody may be prepared by in vitro immunisation of human lymphocytes, followed by transformation of the lymphocytes with Epstein-Barr virus.

For the production of both monoclonal and polyclonal antibodies, the experimental animal is suitably a goat, rabbit, rat or mouse. If desired, the immunogen may be administered as a conjugate in which the immunogen is coupled, for example via a side chain of one of the amino acid residues, to a suitable carrier. The carrier molecule is typically a physiologically acceptable carrier. The antibody obtained may be isolated and, if desired, purified.

An important aspect of the present invention is the use of polypeptides according to the invention in screening methods. The screening methods may be used to identify substances that bind to neurotransmitter transporter polypeptides and in particular which bind to HIPHUM 0000057 such as a ligand or substrate for the polypeptide. Screening methods may also be used to identify agonists or antagonists which may modulate neurotransmitter transporter polypeptide activity, inhibitors or activators of HIPHUM 0000057 activity, and/or agents which up-regulate or down-regulate HIPHUM 0000057 expression.

Any suitable format may be used for the assay. In general terms such screening methods may involve contacting a polypeptide of the invention with a test substance and monitoring for binding of the test substance to the polypeptide or measuring polypeptide activity. A polypeptide of the invention may be incubated with a test substance. Modulation of neurotransmitter transporter activity may be determined. In a preferred aspect, the assay is a cell-based assay. Preferably the assay may be carried out in a single well of a microtitre plate. Assay formats which allow high throughput screening are preferred.

Modulator activity can be determined by contacting cells expressing a polypeptide of the invention with a substance under investigation and by monitoring an effect mediated by the polypeptide. The cells expressing the polypeptide may be in vitro or in vivo. The polypeptide of the invention may be naturally or recombinantly expressed. Preferably, the assay is carried out in vitro using cells expressing recombinant polypeptide. Preferably, control experiments are carried out on cells which do not express the polypeptide of the invention to establish whether the observed responses are the result of activation of the polypeptide.

The binding of a test substance to a polypeptide of the invention can be determined directly. For example, a radiolabelled test substance can be incubated with the polypeptide of the invention and binding of the test substance to the polypeptide can be monitored. Typically, the radiolabelled test substance can be incubated with cell membranes containing the polypeptide until equilibrium is reached. The membranes can then be separated from a non-bound test substance and dissolved in scintillation fluid to allow the radioactive content to be determined by scintillation counting. Non-specific binding of the test substance may also be determined by repeating the experiment in the presence of a saturating concentration of a non-radioactive ligand.

Alternatively, the uptake of a substrate may be monitored using a radiolabelled substance such as GABA. Typically, the assays are carried out in a cellular system, either in a recombinant system or a synaptosomal preparation. The radiolabelled substrate is incubated with the cellular system. Subsequently uptake of the substrate can be determined by lysing the cells and measuring the amount of intracellular labelled substrate or by measuring the amount of labelled substrate remaining in the extracellular medium.

Assays may be carried out using cells expressing HIPHUM 0000057, and incubating such cells with the test substance optionally in the presence of HIPHUM 0000057 ligand. Alternatively an antibody may be used to complex HIPHUM 0000057 and thus mediate HIPHUM 0000057 activity. Test substances may then be added to assess the effect on such activity. Cells expressing HIPHUM 0000057 constitutively may be provided for use in assays for HIPHUM 0000057 function. Additional test substances may be introduced in any assay to look for inhibitors of ligand or substrate binding or inhibitors of HIPHUM 0000057-mediated activity.

Preferably activity is measured in the presence of a neurotransmitter which in a substrate of the neurotransmitter transporter such as GABA.

Assays may also be carried out to identify substances which modify HIPHUM 0000057 polypeptide expression, for example substances which up- or down- regulate expression. Such assays may be carried out for example by using antibodies for HIPHUM 0000057 to monitor levels of HIPHUM 0000057 expression. Other assays which can be used to monitor the effect of a test substance on HIPHUM 0000057 expression include using a reporter gene construct driven by the HIPHUM 0000057 regulatory sequences as the promoter sequence and monitoring for expression of the reporter polypeptide. Further possible assays could utilise membrane fractions from overexpression of HIPHUM 0000057 polypeptide either inX laevis oocytes or cell lines such as HEK293, CHO, COS7 and HeLa cells and assessment of displacement of a radiolabelled ligand.

Additional control experiments may be carried out. Assays may also be carried out using known ligands of other neurotransmitter transporter polypeptides to identify ligands or substrates which are specific for polypeptides of the invention. An assay may be carried out to assess the effect of a test substance on the uptake of a labelled substrate. An assay of the invention may be carried out using a known neurotransmitter transporter agonist or neurotransmitter transporter antagonist to provide a comparison with a compound under test. A neurotransmitter transporter agonist is a substance capable of enhancing neurotransmitter transport. A neurotransmitter transporter antagonist is an inhibitor of neurotransmitter transporter activity.

Suitable test substances which can be tested in the above assays include combinatorial libraries, defined chemical entities and compounds, peptide and peptide mimetics, oligonucleotides and natural product libraries, such as display (e.g. phage display libraries) and antibody products.

Typically, organic molecules will be screened, preferably small organic molecules which have a molecular weight of from 50 to 2500 daltons. Candidate products can be biomolecules including, saccharides, fatty acids, steroids, purines, pyrimidines, derivatives, structural analogs or combinations thereof. Candidate agents are obtained from a wide variety of sources including libraries of synthetic or natural compounds. Known pharmacological agents may be subjected to directed or random chemical modifications, such as acylation, alkylation, esterification, amidification, etc. to produce structural analogs.

Test substances may be used in an initial screen of, for example, 10 substances per reaction, and the substances of these batches which show inhibition or activation tested individually. Test substances may be used at a concentration of from 1 nM to 1000 μM, preferably from 1 μM to 100 μM, more preferably from 1 μM to 10 μM. Preferably, the activity of a test substance is compared to the activity shown by a known activator or inhibitor. A test substance which acts as an inhibitor may produce a 50% inhibition of activity of the polypeptide. Alternatively a test substance which acts as an activator may produce 50% of the maximal activity produced using a known activator.

Another aspect of the present invention is the use of polynucleotides encoding the HIPHUM 0000057 polypeptides of the invention to identify mutations in HIPHUM 0000057 genes which may be implicated in human disorders. Identification of such mutations may be used to assist in diagnosis or susceptibility to such disorders and in assessing the physiology of such disorders. Polynucleotides may also be used in hybridisation studies to monitor for up- or down-regulation of HIPHUM 0000057 expression. Polynucleotides such as SEQ ID NO: 1 or fragments thereof may be used to identify allelic variants, genomic DNA and species variants.

The present invention provides a method for detecting variation in the expressed products encoded by HIPHUM 0000057 genes. This may comprise determining the level of an HIPHUM 0000057 expressed in cells or determining specific alterations in the expressed product. Sequences of interest for diagnostic purposes include, but are not limited to, the conserved portions as identified by sequence similarity and conservation of intron/exon structure. The diagnosis may be performed in conjunction with kindred studies to determine whether a mutation of interest co-segregates with disease phenotype in a family.

Diagnostic procedures may be performed on polynucleotides isolated from an individual or alternatively, may be performed in situ directly upon tissue sections (fixed and/or frozen) of patient tissue obtained from biopsies or resections, such that no nucleic acid purification is necessary. Appropriate procedures are described in, for example, Nuovo, G. J., 1992, “PCR In Situ Hybridization: Protocols And Applications”, Raven Press, NY). Such analysis techniques include, DNA or RNA blotting analyses, single stranded conformational polymorphism analyses, in situ hybridization assays, and polymerase chain reaction analyses. Such analyses may reveal both quantitative aspects of the expression pattern of a HIPHUM 0000057, and qualitative aspects of HIPHUM 0000057 expression and/or composition.

Alternative diagnostic methods for the detection of HIPHUM 0000057 nucleic acid molecules may involve their amplification, e.g. by PCR (the experimental embodiment set forth in U.S. Pat. No. 4,683,202), ligase chain reaction (Barany, 1991, Proc. Natl. Acad. Sci. USA 88:189-193), self sustained sequence replication (Guatelli et al., 1990, Proc. Natl. Acad. Sci. USA 87:1874-1878), transcriptional amplification system (Kwoh et al., 1989, Proc. Natl. Acad. Sci. 15 USA 86:1173-1177), Q-Beta Replicase (Lizardi et al., 1988, Bio/Technology 6:1197) or any other nucleic acid amplification method, followed by the detection of the amplified molecules using techniques well known to those of skill in the art. These detection schemes are especially useful for the detection of nucleic acid molecules if such molecules are present in very low numbers.

Particularly suitable diagnostic methods are chip-based DNA technologies such as those described by Hacia et al., 1996, Nature Genetics 14:441-447 and Shoemaker et al., 1996, Nature Genetics 14:450-456. Briefly, these techniques involve quantitative methods for analyzing large numbers of nucleic acid sequence targets rapidly and accurately. By tagging with oligonucleotides or using fixed probe arrays, one can employ chip technology to segregate target molecules as high density arrays and screen these molecules on the basis of hybridization.

Following detection, the results seen in a given patient may be compared with a statistically significant reference group of normal patients and patients that have HIPHUM 0000057 related pathologies. In this way, it is possible to correlate the amount or kind of HIPHUM 0000057 encoded product detected with various clinical states or predisposition to clinical states.

Another aspect of the present invention is the use of the substances that have been identified by screening techniques referred to above in the treatment of disease states, which are responsive to regulation of neurotransmitter transporter polypeptide activity. The treatment may be therapeutic of prophylactic. The condition of a patient suffering from such a disease state can thus be improved.

A method of treating a subject having a disorder that is responsive to neurotransmitter transporter regulation typically comprises administering to a subject in need thereof, a therapeutically effective amount of a substance identified by a screening method as described herein. A therapeutically effective amount of a substance is an amount which, when administered to a subject in need thereof, improves the condition of the subject. For example, a therapeutically effective amount of a substance may reduce the severity of one or more symptoms of a psychiatric or neurological/neurodegenerative disorder.

In particular, such substances may be used in the treatment of psychiatric disorders, such as bipolar disorders, unipolar depression, anxiety, schizophrenia and psychotic disorders, and neurological/neurodegenerative disorders. Such substances may also be used in the treatment of drug dependence.

Substances identified according to the screening methods outlined above may be formulated with standard pharmaceutically acceptable carriers and/or excipients as is routine in the pharmaceutical art. For example, a suitable substance may be dissolved in physiological saline or water for injections. The exact nature of a formulation will depend upon several factors including the particular substance to be administered and the desired route of administration. Suitable types of formulation are fully described in Remington's Pharmaceutical Sciences, Mack Publishing Company, Eastern Pennsylvania, 17^{th} Ed. 1985, the disclosure of which is included herein of its entirety by way of reference.

The substances may be administered by enteral or parenteral routes such as via oral, buccal, anal, pulmonary, intravenous, intra-arterial, intramuscular, intraperitoneal, topical or other appropriate administration routes.

A therapeutically effective amount of a modulator is administered to a patient. The dose of a modulator may be determined according to various parameters, especially according to the substance used; the age, weight and condition of the patient to be treated; the route of administration; and the required regimen. A physician will be able to determine the required route of administration and dosage for any particular patient. A typical daily dose is from about 0.1 to 50 mg per kg of body weight, according to the activity of the specific modulator, the age, weight and conditions of the subject to be treated, the type and severity of the degeneration and the frequency and route of administration. Preferably, daily dosage levels are from 5 mg to 2 g.

Nucleic acid encoding HIPHUM 0000057 or a variant thereof which inhibits HIPHUM 0000057 activity may be administered to the mammal. Nucleic acid, such as RNA or DNA, and preferably, DNA, is provided in the form of a vector, such as the polynucleotides described above, which may be expressed in the cells of the mammal.

Nucleic acid encoding the polypeptide may be administered by any available technique. For example, the nucleic acid may be introduced by needle injection, preferably intradermally, subcutaneously or intramuscularly. Alternatively, the nucleic acid may be delivered directly across the skin using a nucleic acid delivery device such as particle-mediated gene delivery. The nucleic acid may be administered topically to the skin, or to mucosal surfaces for example by intranasal, oral, intravaginal or intrarectal administration.

Uptake of nucleic acid constructs may be enhanced by several known transfection techniques, for example those including the use of transfection agents. Examples of these agents includes cationic agents, for example, calcium phosphate and DEAE-Dextran and lipofectants, for example, lipofectam and transfectam. The dosage of the nucleic acid to be administered can be altered. Typically the nucleic acid is administered in the range of 1 pg to 1 mg, preferably to 1 pg to 10 g nucleic acid for particle mediated gene delivery and 10 μg to 1 mg for other routes.

The following Examples illustrate the invention.

### EXAMPLE 1

### Characterisation of the Sequence

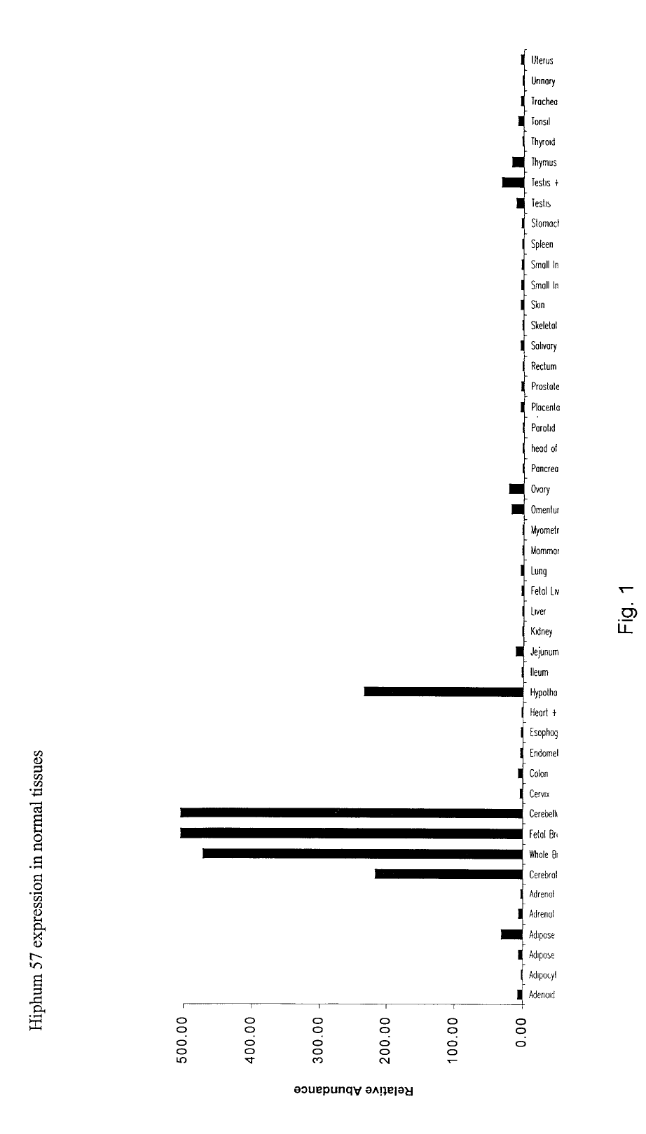

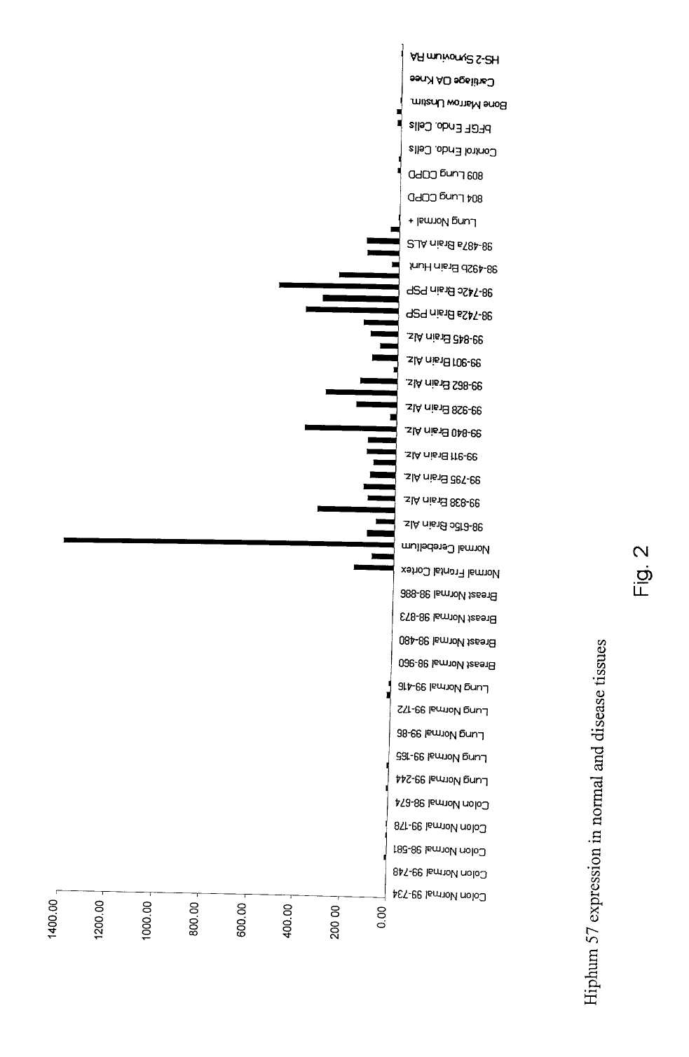

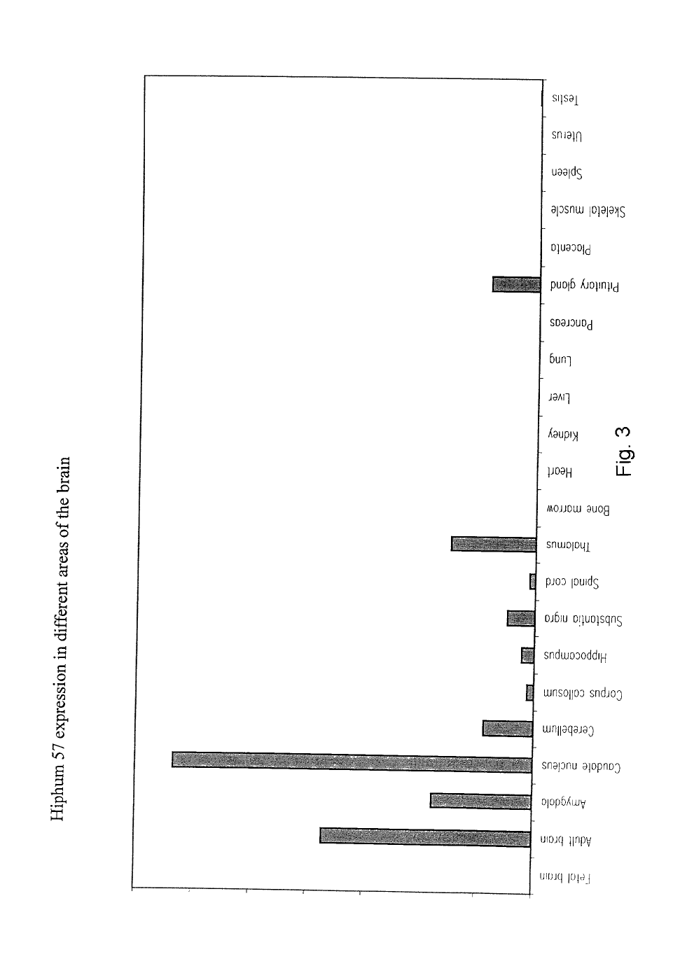

A neurotransmitter transporter polypeptide, designated as HIPHUM 0000057 has been identified. The nucleotide and amino acid sequences of the polypeptide have been determined. These are set out below in SEQ ID NOs: 1 and 2. Suitable primers and probes were designed and used to analyse tissue expression by Taqman™ analysis. HIPHUM 0000057 is specifically expressed in the brain. The highest level of expression is in the candate nucleus. High levels of expression are also found in the amydala and thalamus. These areas of the brain are associated with psychiatric, neurological and neurodegenerative disorders. These results are demonstrated in FIGS. 1-3.

HIPHUM 0000057 has been mapped to the chromosomal region 20q12-q13. The closest known homologue to HIPHUM 0000057 is rat VGAT (SwissProt Accession number 035458), which shares 98% amino acid identity.

### EXAMPLE 2

### Screening for Substances which Exhibit Protein Modulating Activity

For the identification of compounds which modulate transporters, two types of assays are typically being utilised: binding assays with the radiolabeled substrates as ligands and functional uptake assays using these radiolabeled substrates.

In a typical binding assay, the radiolabeled substrate (eg. tritiated substrate) is being incubated with a membrane preparation containing the transporter protein (either recombinant or native) in a suitable incubation buffer (typically in microtiter plates). After a period of incubation (typically between 30 min and 2 hrs) total bound radiolabeled substrate is separated from unbound substrate by means of filtration or any other kind of suitable separation procedure. Nonspecific binding is usually being detected by adding a more than 100-fold excess of unlabeled substrate in parallel experiments. Radioactivity is measured using standard measurement equipment for radioactivity. Specific transporter binding is typically calculated as the difference between total binding minus nonspecific binding. When these kind of experiments are carried out in the presence of compounds to be tested (screening), transporter-modulating properties of such compounds are typically revealed by their radioligand displacing activities. Ki and IC₅₀ values can be calculated in order to analyse their potency.

In a typical functional assay, the uptake of a radiolabeled substrate (eg. tritiated substrate) into an appropriate cellular system is being analysed. Such a cellular system may be a natural system in form of a synaptosomal preparation (for instance) or a recombinant system such as a recombinant cell line expressing the transporter under study. Typically, such a cellular system is incubated with the radiolabeled substrate for a short period (eg. 5 min) in a suitable incubation buffer (typically in microtiter plates). Subsequently, the incubation buffer is removed from the cells and cells are lysed by the addition of a lysing agent such as a detergent. Radioactivity of the cell lysates is then analysed using standard techniques. The amount of radioactivity detected in the cell lysate is a measure of the transport activity. If these kind of experiments are carried out in the presence of compounds to be tested (screening), uptake inhibitors are identified by virtue of their uptake reducing capacity. On the other hand, uptake stimulators may be identified by their uptake increasing capacity. Respective potencies of such compounds can be calculated and expressed as IC₅₀ values for instance.

1. An isolated neurotransmitter transporter polypeptide comprising the amino acid sequence of SEQ ID NO: 2.

2. The isolated polypeptide of claim 1 wherein the polypeptide consists of the amino acid sequence of SEQ ID NO: 2.

3. An isolated neurotransmitter transporter polypeptide comprising a variant of the polypeptide comprising the amino acid sequence of SEQ ID NO: 2 which variant shows a function of the polypeptide of claim 1.

4. An isolated neurotransmitter transporter polypeptide comprising a fragment of the polypeptide comprising the amino acid sequence set forth in SEQ ID NO: 2, which fragment shows a function of the polypeptide of claim 1.

5. A polypeptide according to claim 3 wherein the variant has at least 80% identity to the amino acid sequence set forth in SEQ ID NO: 2.

6. An isolated polynucleotide encoding a polypeptide according to claim 1.

7. An isolated polynucleotide encoding a polypeptide according to claim 2.

8. An isolated polynucleotide encoding a polypeptide according to claim 3.

9. An isolated polynucleotide encoding a polypeptide according to claim 4.

10. An isolated polynucleotide encoding a polypeptide according to claim 5.

11. An isolated polynucleotide according to claim 6 which is a cDNA sequence.

12. An isolated polynucleotide encoding a neurotransmitter transporter polypeptide which polynucleotide compri ses the nucleic acid sequence set forth in SEQ ID NO: 1 or a sequence complementary thereto.

13. An isolated polynucleotide comprising a polynucleotide which hybridises under stringent conditions to a polynucleotide of claim 11.

14. An isolated polynucleotide comprising a polynucleotide comprising a nucleic acid sequence having at least 80% identity to a polynucleotide of claim 11.

15. An isolated polynucleotide consisting essentially of the nucleic acid sequence of SEQ ID NO: 1 or a sequence complementary thereto.

16. An isolated polynucleotide comprising a polynucleotide which hybridises under stringent conditions to a polynucleotide of claim 15.

17. An expression vector comprising a polynucleotide according to claim 6.

18. A host cell comprising an expression vector according to claim 17.

19. An antibody specific for a polypeptide according to claim 1.

20. An antibody specific for a polypeptide according to claim 4.

21. A method for the identification of a substance that modulates neurotransmitter transporter activity, which method comprises:
(i) contacting a test substance with a polypeptide according to claim 1 or a fragment thereof, and
(ii) determining the effect of the test substance on the activity of the said polypeptide, thereby to determine whether the test substance modulates neurotransmitter transporter activity.

22. A method according to claim 21 wherein the polypeptide is expressed in a cell.

23. A method for the identification of a substance that modulates neurotransmitter transporter polypeptide expression, which method comprises:
(i) contacting a test substance with a polynucleotide according to claim 6 or a fragment thereof, and
(ii) determining the effect of the test substance on the expression of the said polynucleotide, thereby to determine whether the test substance modulates neurotransmitter transporter polypeptide expression.

24. An isolated substance which modulates neurotransmitter transporter polypeptide activity and which is identifiable by a method according to claim 21.

25. An isolated substance which modulates neurotransmitter transporter polypeptide expression and which is identifiable by a method according to claim 23.

26. A method of treating a subject having a disorder that is responsive to neurotransmitter transporter polypeptide modulation, which method comprises administering to said subject an effective amount of a substance according to claim 24.

27. A method according to claim 26 wherein the disorder is selected from psychiatric disorders and neurological/neurodegenerative disorders.

28. A method according to claim 27 wherein the psychiatric disorder is selected from bipolar disorder, unipolar depression, anxiety, schizophrenia and psychotic disorders.

29. A method of producing a isolated neurotransmitter receptor polypeptide, which method comprises maintaining a host cell as defined in claim 18 under conditions suitable for obtaining expression of the polypeptide, and isolating the said polypeptide.

30. A method of treating a subject having a disorder that is responsive to neurotransmitter transporter polypeptide modulation, comprising administering to said subject a therapeutically effective amount of a modulator identified by a method according to claim 23.

31. A method according to claim 30 wherein the disorder is selected from psychiatric disorders and neurological/neurodegenerative disorders.

32. A method according to claim 31 wherein the psychiatric disorder is selected from bipolar disorder, unipolar depression, anxiety, schizophrenia and psychotic disorders.

33. A composition comprising a modulator of the activity of a protein having the amino acid sequence set forth in SEQ ID NO: 2, which is identified by a method according to claim 21.

34. A composition comprising a modulator of the expression of a protein having the amino acid sequence set forth in SEQ ID NO: 2, which is identified by a method according to claim 23.
